(19) United States
(12) Patent Application Publication
(10) Pub. No.: US 2006/0035867 A1
(43) Pub. Date: Feb. 16, 2006

(54) Methods of treating cancer and the pain associated therewith using endothelin antagonists

(76) Inventors: Todd J. Janus, Gurnee, IL (US); Robert J. Padley, Lake Bluff, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/243,320

(22) Filed: Oct. 4, 2005

Related U.S. Application Data
(63) Continuation of application No. 09/923,616, filed on Aug. 6, 2001, now abandoned.
(60) Provisional application No. 60/223,486, filed on Aug. 7, 2000.

(51) Int. Cl. A61K 38/09; A61K 31/66; A61K 31/59; A61K 31/56; A61K 31/496

(52) U.S. Cl. 514/102; 514/15; 514/167; 514/171; 514/254.07

(57) ABSTRACT

The instant invention is directed to methods for the inhibition of bone metastases, methods for the prevention of growth of new metastases, methods for the inhibition of bone turnover, and methods for the prevention of bone loss in patients, including cancer patients, using an endothelin ET-A receptor antagonist.

This application is a continuation of U.S. application Ser. No. 09/923,616, filed Aug. 6, 2001 which claims priority to U.S. Provisional Application Ser. No. 60/223,486, filed Aug. 7, 2000.

### FIELD OF THE INVENTION

The instant invention is directed to methods for the inhibition of bone metastases, methods for the prevention of growth of new metastases, methods for the inhibition of bone turnover, and methods for the prevention of bone loss in patients, including cancer patients, using an endothelin ET-A receptor antagonist.

### BACKGROUND OF THE INVENTION

Endothelin (ET), a 21 amino acid peptide, is produced by enzymatic cleavage of a precursor peptide by an endothelin converting enzyme. First discovered in vascular endothelial cells, ET and ET/ET receptor binding are now known to modulate smooth muscle tone, blood flow, cell proliferation and differentation, protein synthesis, and metabolic function in a variety of tissues and cell types such as ovary, prostate, skin, and brain.

ET/ET receptor binding has been shown to constrict arteries and veins; increase mean arterial blood pressure; decrease incardiac output; increase cardiac contractility in vitro; stimulate mitogenesis in vascular smooth muscle cells in vitro; contract non-vascular smooth muscle such as guinea pig trachea, human urinary bladder strips and rat uterus in vitro; increase airway resistance in vivo; induce formation of gastric ulcers; stimulate release of atrial natriuretic factor in vitro and in vivo; increase plasma levels of vasopressin, aldosterone, and catecholamines; inhibit release of renin in vitro; and stimulate release of gonadotropins in vitro.

ET/ET receptor binding also causes vasoconstriction on vascular smooth muscle (Nature 332 411 (1988), FEBS Letters 231 440 (1988) and Biochem. Biophys. Res. Commun. 154 868 (1988)). In fact, an anti-ET antibody has been shown to ameliorate adverse effects of renal ischemia on renal vascular resistance and glomerular filtration rate (J. Clin. Invest. 83 1762 (1989)). In addition, an anti-ET antibody attenuated both the nephrotoxic effects of intravenously administered cyclosporin (Kidney Int. 37 1487 (1990)) and the infarct size in a coronary artery ligation-induced myocardial infarction model (Nature 344 114 (1990)).

A nonpeptide ET antagonist prevents post-ischaemic renal vasoconstriction in rats, prevents the decrease in cerebral blood flow due to subarachnoid hemorrhage in rats, and decreases MAP in sodium-depleted squirrel monkeys when dosed orally (Nature 365: 759-761 (1993)). A similar effect of an ET antagonist on arterial calibera has also been recently reported (Biochem. Biophys. Res. Comm., 195: 969-75 (1993).

An ET receptor antagonist reduced injury in a rat model of colitis (EUR. J. Pharmacol. 1996, 309, 261-269) and prevented ischemia-reperfusion injury in kidney transplantation (Transplant Int 1996, 9, 201-207). The use of ET antagonists in the treatment of angina, pulmonary hypertension, Raynaud's disease, and migraine has also been suggested (Drugs 1996, 51, 12-27). In malignant growth disorders, ET and its growth-promoting effects have been best characterized in prostate cancer, (Nature Medicine 1995, 1, 944-949) wherein ET acts as a modulator in osteoblastic bone lesion (UROLOGY 53:1063-1069, 1999).

Given the results from these and other reports which illuminate the role of ET/ET receptor binding in disease states, and the knowledge that blocking ET/ET receptor binding results in improvement or reversal of endothelin-induced disease states, agents which antagonize ET/ET receptor binding activity, designated as ET receptor antagonists, can provide substantial benefit in many therapeutic areas.

### SUMMARY OF THE INVENTION

In one embodiment of the instant invention, therefore, is disclosed a method for inhibiting bone metastases in a patient which comprises administering to the patient in need thereof a therapeutically effective amount of an endothelin ET-A receptor antagonist.

In another embodiment of the invention is disclosed a method for preventing new bone metastases in a patient which comprises administering to the patient in need thereof a therapeutically effective amount of an endothelin ET-A receptor antagonist.

In another embodiment of the instant invention, therefore, is disclosed a method for inhibiting metastatic growth in a patient which comprises administering to the patient in need thereof a therapeutically effective amount of an endothelin ET-A receptor antagonist.

In another embodiment of the invention is disclosed a method for inhibiting bone loss in a patient which comprises administering to the patient in need thereof a therapeutically effective amount of an endothelin ET-A receptor antagonist.

In another embodiment of the instant invention, is disclosed a method for inhibiting bone turnover in a patient which comprises administering to the patient in need thereof a therapeutically effective amount of an endothelin ET-A receptor antagonist.

In another embodiment of the invention is disclosed a method for the reduction of cancer related pain in a patient in need thereof which comprises administering to the patient a therapeutically effective amount of an endothelin ET-A receptor antagonist.

In another embodiment of the instant invention is disclosed therapeutically acceptable formulations of an endothelin ET-A receptor antagonist, optionally in the presence of a co-therapeutic agent, for use in these methods.

### BRIEF DESCRIPTION OF THE DRAWINGS

FIG. 1 illustrates levels of interleukin-6 (IL-6) in a subject population treated with a placebo or 2.5 mg or 10 mg ABT-627.

FIG. 2 illustrates levels of prostate specific antigen (PSA) in a subject population treated with a placebo or 2.5 mg or 10 mg of ABT-627.

FIG. 3 illustrates VAS score levels relating to pain assessment in a subject population treated with a placebo or 2.5 mg or 10 mg of ABT-627.

FIG. 4 illustrates crosslinked N-telopeptides (degradation) in a subject population treated with a placebo or 10 mg ABT-627.

FIG. 5 illustrates bone alkaline phosphatase (BAP)(formation) in a subject population treated with a placebo or 10 mg ABT-627.

FIG. 6 illustrates skeletal involvement in a subject population treated with a placebo or 10 mg ABT-627.

FIG. 7 illustrates acid phosphatase levels in a subject population treated with a placebo or 10 mg ABT-627.

### DETAILED DESCRIPTION OF THE INVENTION

Endothelin receptor antagonists are employed in the practice of the instant invention. Endothelins are a family of peptides mainly synthesized and released by endothelial cells. The term “endothelin” refers to a family of homologous 21-amino acid peptides found in 3 distinct isoforms: ET-1, ET-2, and ET-3.

The term “endothelin ET-A receptor antagonist” includes both compounds which antagonize the ET-A receptor in a selective manner, as well as compounds which antagonize the ET-A receptor in a non-selective manner. An example of the latter type of compound would be a compound that antagonizes the ET-A receptor and also antagonizes the ET-B receptor.

The term “primary cancer” means cancer in a specific tissue, which is first in time or in order of development. Primary cancers include, but are not limited to, breast, prostate, lung, kidney, thyroid, brain, heart, intestine, ovary, myeloma, lymphoma, sarcoma, and osteosarcoma.

The term “cancer-related pain” includes pain which arises from direct invasion or expansion of a tumor into tissue, such as bone or nerve; pain which arises from the consequences of tumor invasion or expansion, such as bone collapse due to cancer erosion or secretion of noxious agents which modulate or produce pain; and pain mediated by ischemia, i.e. reduced blood flow.

Specifically, a compound of formula I may be employed in the practice of the instant invention 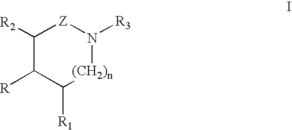
wherein
R is —(CH₂)ₘ—W;
Z is selected from —C(R₁₈)(R₁₉)— and —C(O)—;
R₁ and R₂ are independently selected from hydrogen, loweralkyl, alkenyl, alkynyl, alkoxyalkyl, alkoxycarbonylalkyl, hydroxyalkyl, haloalkyl, haloalkoxyalkyl, alkoxyalkoxyalkyl, thioalkoxyalkoxyalkyl, cycloalkyl, cycloalkylalkyl, aminocarbonylalkyl, alkylaminocarbonylalkyl, dialkylaminocarbonylalkyl, aminocarbonylalkenyl, alkylaminocarbonylalkenyl, dialkylaminocarbonylalkenyl, hydroxyalkenyl, aryl, arylalkyl, aryloxyalkyl, arylalkoxyalkyl, (N-alkanoyl-N-alkyl)aminoalkyl, alkylsulfonylamidoalkyl, heterocyclic, (heterocyclic)alkyl, and (Rₐₐ)(R_{bb})N—R_{cc}—,
with the proviso that one or both of R₁ and R₂ is other than hydrogen;
R₃ is selected from R₄—C(O)—R₅—, R₄—R₅ₐ—, R₄—C(O)—R₅—N(R₆)—, R₆—S(O)₂—R₇—R₂₆—S(O)—R₂₇—, R₂₂—O—C(O)—R₂₃—, loweralkyl, alkenyl, alkynyl, cycloalkyl, cycloalkylalkyl, aryl, arylalkyl, aryloxyalkyl, heterocyclic, (heterocyclic)alkyl, alkoxyalkyl, alkoxyalkoxyalkyl, and R₁₃—C(O)—CH(R₁₄)—;
R₄ and R₆ are independently selected from (R₁₁)(R₁₂)N—, loweralkyl, alkenyl, alkynyl, cycloalkyl, cycloalkylalkyl, aryl, arylalkyl, heterocyclic, (heterocyclic)alkyl, alkoxyalkyl, hydroxyalkyl, haloalkyl, haloalkenyl, haloalkoxyalkyl, haloalkoxy, alkoxyhaloalkyl, alkylaminoalkyl, dialkylaminoalkyl, alkoxy, and 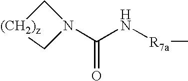
R₅ is selected from a covalent bond, alkylene, alkenylene, —N(R₂₀)—R₈—, —R₈ₐ—N(R₂₀)—R₈—, —O—R₉—, and —R₉ₐ—O—R₉—;
R₆ is selected from loweralkyl, haloalkyl, alkoxyalkyl, haloalkoxyalkyl, aryl or arylalkyl;
R₇ is a covalent bond, alkylene, alkenylene —N(R₂₁)—R₁₀—, and —R₁₀ₐ—N(R₂₁)—R₁₀—;
R₈ is selected from alkylene and alkenylene;
R₉ is alkylene;
R₁₀ is selected from alkylene and alkenylene;
R₁₁ and R₁₂ are independently selected from hydrogen, loweralkyl, haloalkyl, alkoxyalkyl, haloalkoxyalkylalkenyl, alkynyl, cycloalkyl, cycloalkylalkyl, aryl, heterocyclic, arylalkyl, (heterocyclic)alkyl, hydroxyalkyl, alkoxy, aminoalkyl, trialkylaminoalkyl, alkylaminoalkyl, dialkylaminoalkyl, and carboxyalkyl;
R₁₃ is selected from amino, alkylamino and dialkylamino;
R₁₄ is selected from aryl and R₁₅—C(O)—;
R₁₅ is selected from amino, alkylamino and dialkylamino;
R₁₆ is selected from loweralkyl, haloalkyl, aryl and dialkylamino;
R₁₇ is loweralkyl;
R₁₈ and R₁₉ are independently selected from hydrogen and loweralkyl;
R₂₀ is selected from hydrogen, loweralkyl, alkenyl, haloalkyl, alkoxyalkyl, haloalkoxyalkyl, cylcoalkyl and cycloalkylalkyl;
R₂₁ is selected from hydrogen, loweralkyl, alkenyl, haloalkyl, alkoxyalkyl, haloalkoxyalkyl, aryl and arylalkyl;
R₂₂ is selected from a carboxy protecting group and heterocyclic;
R₂₃ is selected from covalent bond, alkylene, alkenylene and —N(R₂₄)—R₂₅—;
R₂₄ is selected from hydrogen and loweralkyl;
R₂₅ is alkylene;
R₂₆ is selected from loweralkyl, haloalkyl, alkenyl, alkynyl, cycloalkyl, cycloalkylalkyl, aryl, arylalkyl, heterocyclic, (heterocyclic)alkyl, alkoxyalkyl and alkoxy-substituted haloalkyl;
R₂₇ is selected from alkylene and alkenylene;
R₅ₐ is selected from alkylene and alkenylene;
R₇ₐ is alkylene;
R₈ₐ is selected from alkylene and alkenylene;
R₉ₐ is alkylene;
R₁₀ₐ is selected from alkylene and alkenylene;
Rₐₐ is selected from aryl and arylalkyl;
R_{bb} is selected from hydrogen and alkanoyl;
R_{cc} is alkylene;
m is 0-6;
n is 0 or 1;
z is 0-5;
E is selected from hydrogen, loweralkyl and arylalkyl;
G is selected from hydrogen and a carboxy protecting group; and
W is selected from —C(O)₂-G; —PO₃H₂, —P(O)(OH)(E), —CN, —C(O)NHR₁₇, alkylaminocarbonyl, dialkylaminocarbonyl, tetrazolyl, hydroxy, alkoxy, sulfonamido, —C(O)NHS(O)₂R₁₆, —S(O)₂NHC(O)R₁₆, 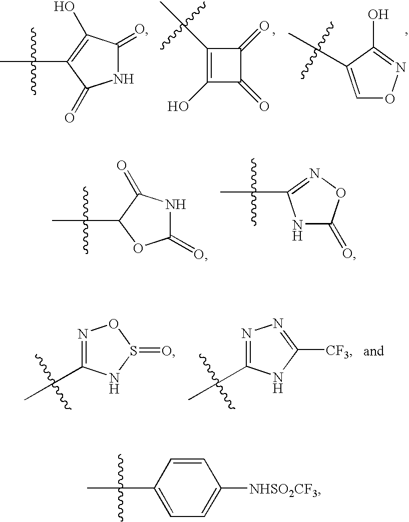
   or a pharmaceutically acceptable salt thereof.

A preferred embodiment of the a compound of formula I is a compound of formula II 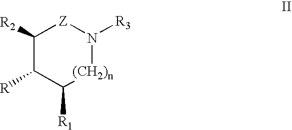
wherein the substituents —R₂, —R and —R₁ exist in a trans,trans relationship and Z, n, R, R₁, R₂, and R₃ are as defined above.

Compounds of formulas I and II are endothelin antagonists, specifically ET_{A}-selective endothelin antagonists.

Another preferred embodiment of the invention is a compound of formula I or II wherein n is 0 and Z is —CH₂—.

Another preferred embodiment of the invention is a compound of formula I or II wherein n is 1 and Z is —CH₂—.

Another preferred embodiment of the invention is a compound of formula I or II wherein n is 0, Z is —CH₂—, and R₃ is R₄—C(O)—R₅—, R₆—S(O)₂—R₇— or R₂₆—S(O)—R₂₇—wherein R₄, R₅, R₆, R₇, R₂₆ and R₂₇ are as defined above.

Another preferred embodiment of the invention is a compound of formula I or II wherein n is 0, Z is —CH₂—, and R₃ is alkoxyalkyl or alkoxyalkoxyalkyl.

A more preferred embodiment of the invention is a compound of formula I or II wherein n is 0, Z is —CH₂—, and R₃ is R₄—C(O)—R₅— wherein R₄ is (R₁₁)(R₁₂)N— as defined above and R₅ is alkylene or R₃ is R₆—S(O)₂—R₇— or R₂₆—S(O)—R₂₇— wherein R₇ is alkylene, R₂₇ is alkylene and R₆ and R₂₆ are defined as above.

Another more preferred embodiment of the invention is a compound of formula I or II wherein n is 0, Z is —CH₂— and R₃ is R₄—C(O)—N(R₂₀)—R₈— or R₆—S(O)₂—N(R₂₁)—R₁₀— wherein R₈ and R₁₀ are alkylene and R₄, R₆, R₂₀ and R₂₁ are defined as above.

An even more preferred embodiment of the invention is a compound of formula I or II wherein n is 0, R is tetrazolyl or —C(O)₂-G wherein G is hydrogen or a carboxy protecting group or R is tetrazolyl or R is —C(O)—NHS(O)₂R₁₆ wherein R₁₆ is loweralkyl, haloalkyl or aryl, Z is —CH₂—; R₁ and R₂ are independently selected from (i) loweralkyl, (ii) cycloalkyl, (iii) substituted aryl wherein aryl is phenyl substituted with one, two or three substituents independently selected from loweralkyl, alkoxy, halo, alkoxyalkoxy and carboxyalkoxy, (iv) substituted or unsubstituted heterocyclic, (v) alkenyl, (vi) heterocyclic (alkyl), (vii) arylalkyl, (viii) aryloxyalkyl, (ix) (N-alkanoyl-N-alkyl)aminoalkyl and (x) alkylsulfonylamidoalkyl, and R₃ is R₄—C(O)—R₅—wherein R₄ is (R₁₁)(R₁₂)N— wherein R₁₁ and R₁₂ are independently selected from loweralkyl, haloalkyl, alkoxyalkyl, haloalkoxyalkyl, aryl, arylalkyl, heterocyclic, hydroxyalkyl, alkoxy, aminoalkyl, and trialkylaminoalkyl, and R₅ is alkylene; or R₃ is R₄—C(O)—N(R₂₀)—R₈— or R₆—S(O)₂—N(R₂₁)—R₁₀— wherein R₄ is loweralkyl, aryl, alkoxy, alkylamino, aryloxy or arylalkoxy and R₆ is loweralkyl, haloalkyl, alkoxyalkyl, haloalkoxyalkyl, aryl or arylalkyl, R₈ and R₁₀ are alkylene and R₂₀ and R₂₁ are loweralkyl; or R₃ is R₆—S(O)₂—R₇— or R₂₆—S(O)—R₂₇— wherein R₆ is loweralkyl or haloalkyl, R₇ is alkylene, R₂₆ is loweralkyl and R₂₇ is alkylene.

A yet more preferred embodiment of the invention is a compound of formula I or II wherein n is 0, R is —C(O)₂-G wherein G is hydrogen or a carboxy protecting group, tetrazolyl or —C(O)—NHS(O)₂R₁₆ wherein R₁₆ is loweralkyl, haloalkyl or aryl, Z is —CH₂—, R₁ is (i) loweralkyl, (ii) alkenyl, (iii) alkoxyalkyl, (iv) cycloalkyl, (v) phenyl, (vi) pyridyl, (vii) furanyl, (viii) substituted or unsubstituted 4-methoxyphenyl, 4-fluorophenyl, 3-fluorophenyl, 4-ethoxyphenyl, 4-ethylphenyl, 4-methylphenyl, 4-trifluoromethylphenyl, 4-pentafluoroethylphenyl, 3-fluoro-4-methoxyphenyl, 3-fluoro-4-ethoxyphenyl, 2-fluorophenyl, 4-methoxymethoxyphenyl, 4-hydroxyphenyl, 4-t-butylphenyl, 1,3-benzodioxolyl, 1,4-benzodioxanyl or dihydrobenzofuranyl wherein the substituent is selected from alkoxy, alkoxyalkoxy and carboxyalkoxy, (ix) heterocyclic (alkyl), (x) arylalkyl, (xi) aryloxyalkyl, (xii) (N-alkanoyl-N-alkyl)aminoalkyl, or (xiii) alkylsulfonylamidoalkyl, R₂ is substituted or unsubstituted 1,3-benzodioxolyl, 7-methoxy-1,3-benzodioxolyl, 1,4-benzodioxanyl, 8-methoxy-1,4-benzodioxanyl, dihydrobenzofuranyl, benzofurnayl, 4-methoxyphenyl, dimethoxyphenyl, fluorophenyl or difluorophenyl and R₃ is R₄—C(O)—N(R₂₀)—R₈— or R₆—S(O)₂—N(R₂₁)—R₁₀— wherein R₈ and R₁₀ are alkylene, R₂₀ and R₂₁ are loweralkyl, R₄ is loweralkyl, aryl, alkoxy, alkylamino, aryloxy or arylalkoxy and R₆ is loweralkyl, haloalkyl, alkoxyalkyl, aryl or arylalkyl.

Another yet more preferred embodiment of the invention is a compound of formula I or II wherein n is 0, R is —C(O)₂-G wherein G is hydrogen or a carboxy protecting group, tetrazolyl or —C(O)—NHS(O)₂R₁₆ wherein R₁₆ is loweralkyl, haloalkyl or aryl, Z is —CH₂—, R₁ is (i) loweralkyl, (ii) alkenyl, (iii) alkoxyalkyl, (iv) cycloalkyl, (v) phenyl, (vi) pyridyl, (vii) furanyl, (viii) substituted or unsubstituted 4-methoxyphenyl, 4-fluorophenyl, 3-fluorophenyl, 4-ethoxyphenyl, 4-ethylphenyl, 4-methylphenyl, 4-trifluoromethylphenyl, 4-pentafluoroethylphenyl, 3-fluoro-4-methoxyphenyl, 3-fluoro-4-ethoxyphenyl, 2-fluorophenyl, 4-methoxymethoxyphenyl, 4-hydroxyphenyl, 4-t-butylphenyl, 1,3-benzodioxolyl, 1,4-benzodioxanyl or dihydrobenzofuranyl wherein the substituent is selected from alkoxy, alkoxyalkoxy and carboxyalkoxy, (ix) heterocyclic (alkyl), (x) arylalkyl, (xi) aryloxyalkyl, (xii) (N-alkanoyl-N-alkyl)aminoalkyl, or (xiii) alkylsulfonylamidoalkyl, R₂ is substituted or unsubstituted 1,3-benzodioxolyl, 7-methoxy-1,3-benzodioxolyl, 1,4-benzodioxanyl, 8-methoxy-1,4-benzodioxanyl, dihydrobenzofuranyl, benzofurnayl, 4-methoxyphenyl, dimethoxyphenyl, fluorophenyl or difluorophenyl and R₃ is R₄—C(O)—R₅— wherein R₅ is alkylene and R₄ is (R₁₁)(R₁₂)N— wherein R₁₁ and R₁₂ are independently selected from loweralkyl, haloalkyl, alkoxyalkyl, haloalkoxyalkyl, aryl, arylalkyl, heterocyclic, hydroxyalkyl, alkoxy, aminoalkyl, and trialkylaminoalkyl.

Another yet more preferred embodiment of the invention is a compound of formula I or II wherein n is 0, R is —C(O)₂-G wherein G is hydrogen or a carboxy protecting group, tetrazolyl or —C(O)—NHS(O)₂R₁₆ wherein R₁₆ is loweralkyl, haloalkyl or aryl, Z is —CH₂—, R₁ is (i) loweralkyl, (ii) alkenyl, (iii) heterocyclic(alkyl), (iv) aryloxyalkyl, (v) arylalkyl, (vi) aryl, (vii) (N-alkanoyl-N-alkyl)aminoalkyl, or (viii) alkylsulfonylamidoalkyl, R₂ is substituted or unsubstituted 1,3-benzodioxolyl, 7-methoxy-1,3-benzodioxolyl, 1,4-benzodioxanyl, 8-methoxy-1,4-benzodioxanyl, dihydrobenzofuranyl, benzofurnayl, 4-methoxyphenyl, dimethoxyphenyl, fluorophenyl or difluorophenyl wherein the substituent is selected from loweralkyl, alkoxy and halogen and R₃ is R₄—C(O)—R₅— wherein R₅ is alkylene and R₄ is (R₁₁)(R₁₂)N— wherein R₁₁ is loweralkyl and R₁₂ is aryl, arylalkyl, hydroxyalkyl, alkoxy, aminoalkyl, trialkylaminoalkyl, or heterocyclic.

Another yet more preferred embodiment of the invention is a compound of formula I or II wherein n is 0, R is —C(O)₂-G wherein G is hydrogen or a carboxy protecting group, tetrazolyl or —C(O)—NHS(O)₂R₁₆ wherein R₁₆ is loweralkyl, haloalkyl or aryl, Z is —CH₂—, R₁ is (i) loweralkyl, (ii) alkenyl, (iii) heterocyclic (alkyl), (iv) aryloxyalkyl, (v) arylalkyl, (vi) (N-alkanoyl-N-alkyl)aminoalkyl, or (vii) alkylsulfonylamidoalkyl, (vii) phenyl, or (ix) substituted or unsubstituted 4-methoxyphenyl, 3-fluoro-4-methoxyphenyl, 3-fluorophenyl, 3-fluoro-4-ethoxyphenyl, 2-fluorophenyl, 4-methoxymethoxyphenyl, 1,3-benzodioxolyl, 1,4-benzodioxanyl or dihydrobenzofuranyl wherein the substituent is selected from loweralkyl, haloalkyl, alkoxy, alkoxyalkoxy and carboxyalkoxy, R₂ is substituted or unsubstituted 1,3-benzodioxolyl, 7-methoxy-1,3-benzodioxolyl, 1,4-benzodioxanyl, 8-methoxy-1,4-benzodioxanyl, dihydrobenzofuranyl, 4-methoxyphenyl, dimethoxyphenyl, fluorophenyl or difluorophenyl wherein the substituent is selected from loweralkyl, alkoxy and halogen and R₃ is R₆—S(O)₂—N(R₂₁)—R₁₀— wherein R₁₀ is alkylene, R₆ is loweralkyl, haloalkyl, alkoxyalkyl, haloalkoxyalkyl, aryl or arylalkyl and R₂₁ is loweralkyl, haloalkyl, alkoxyalkyl, haloalkoxyalkyl, aryl or arylalkyl.

Another yet more preferred embodiment of the invention is a compound of formula I or II wherein n is 0, R is —C(O)₂-G wherein G is hydrogen or a carboxy protecting group, tetrazolyl or —C(O)—NHS(O)₂R₁₆ wherein R₁₆ is loweralkyl, haloalkyl or aryl, Z is —CH₂—, R₁ is (i) substituted or unsubstituted 4-methoxyphenyl, 3-fluoro-4-methoxyphenyl, 3-fluorophenyl, 3-fluoro-4-ethoxyphenyl, 4-methoxymethoxyphenyl, 1,3-benzodioxolyl or 1,4-benzodioxanyl wherein the substituent is selected from loweralkyl, haloalkyl, alkoxy and alkoxyalkoxy, (ii) loweralkyl, (iii) alkenyl, (iv) heterocyclic (alkyl), (v) aryloxyalkyl, (vi) arylalkyl, (vii) (N-alkanoyl-N-alkyl)aminoalkyl, (viii) alkylsulfonylamidoalkyl, or (ix) phenyl, R₂ is substituted or unsubstituted 1,3-benzodioxolyl, 7-methoxy-1,3-benzodioxolyl, 1,4-benzodioxanyl, 8-methoxy-1,4-benzodioxanyl, dihydrobenzofuranyl, 4-methoxyphenyl, dimethoxyphenyl, fluorophenyl or difluorophenyl wherein the substituent is selected from loweralkyl, alkoxy and halogen and R₃ is alkoxycarbonyl or R₆—S(O)₂—N(R₂₁)—R₁₀— wherein R₁₀ is alkylene, R₆ is loweralkyl, haloalkyl, alkoxyalkyl or haloalkoxyalkyl and R₂₁ is loweralkyl, haloalkyl, alkoxyalkyl or haloalkoxyalkyl.

Another yet more preferred embodiment of the invention is a compound of formula I or II wherein n is 0, R is —C(O)₂-G wherein G is hydrogen or a carboxy protecting group, tetrazolyl or —C(O)—NHS(O)₂R₁₆ wherein R₁₆ is loweralkyl or haloalkyl, Z is —CH₂—, R₁ is loweralkyl,alkenyl, heterocyclic (alkyl), aryloxyalkyl, aryalkyl, aryl, (N-alkanoyl-N-alkyl)aminoalkyl, or alkylsulfonylamidoalkyl, and R₃ is R₄—C(O)—R₅— wherein R₅ is alkylene and R₄ is (R₁₁)(R₁₂)N— wherein R₁₁ and R₁₂ are independently selected from alkyl, aryl, hydroxyalkyl, alkoxy, aminoalkyl, trialkylaminoalkyl, and heterocyclic.

A still more preferred embodiment of the invention is a compound of formula I or II wherein n is 0, R is —C(O)₂-G wherein G is hydrogen or a carboxy protecting group, tetrazolyl or —C(O)—NHS(O)₂R₁₆ wherein R₁₆ is loweralkyl or haloalkyl, Z is —CH₂—, R₁ is substituted or unsubstituted 4-methoxyphenyl, 4-fluorophenyl, 2-fluorophenyl, 4-methylphenyl, 4-trifluoromethylphenyl, 4-pentafluoroethylphenyl, 4-methoxymethoxyphenyl, 4-hydroxyphenyl, 4-ethylphenyl, 1,3-benzodioxolyl, 1,4-benzodioxanyl or dihydrobenzofuranyl wherein the substituent is selected from alkoxy, alkoxyalkoxy and carboxyalkoxy, (ii) loweralkyl, (iii) alkenyl, (iv) heterocyclic (alkyl), (v) aryloxyalkyl, (vi) arylalkyl, (vii) (N-alkanoyl-N-alkyl)aminoalkyl, (viii) alkylsulfonylamidoalkyl, or (ix) phenyl, R₂ is 1,3-benzodioxolyl, 1,4-benzodioxanyl, dihydrobenzofuranyl, benzofuranyl, 4-methoxyphenyl, dimethoxyphenyl, fluorophenyl or difluorophenyl and R₃ is R₄—C(O)—R₅— wherein R₅ is alkylene and R₄ is (R₁₁)(R₁₂)N— wherein R₁₁ and R₁₂ are independently selected from loweralkyl, aryl, arylalkyl, hydroxyalkyl, alkoxy, aminoalkyl, trialkylaminoalkyl, or heterocyclic.

Another still more preferred embodiment of the invention is a compound of formula I or II wherein n is 0, R is —C(O)₂-G wherein G is hydrogen or a carboxy protecting group, tetrazolyl or —C(O)—NHS(O)₂R₁₆ wherein R₁₆ is loweralkyl or haloalkyl, Z is —CH₂—, R₁ is loweralkyl, alkenyl, heterocyclic (alkyl), aryloxyalkyl, arylalkyl, (N-alkanoyl-N-alkyl)aminoalkyl, alkylsulfonylamidoalkyl, phenyl, or alkoxyalkyl, R₂ is 1,3-benzodioxolyl, 1,4-benzodioxanyl, dihydrobenzofuranyl, benzofuranyl, 4-methoxyphenyl, dimethoxyphenyl, fluorophenyl or difluorophenyl and R₃ is R₄—C(O)—R₅— wherein R₅ is alkylene and R₄ is (R₁₁)(R₁₂)N-wherein R₁₁ and R₁₂ are independently selected from loweralkyl, aryl, arylalkyl, hydroxyalkyl, alkoxy, aminoalkyl, trialkylaminoalkyl, or heterocyclic.

A most highly preferred embodiment of the invention is a compound of formula I or II wherein n is 0, R is —C(O)₂-G wherein G is hydrogen or a carboxy protecting group, Z is —CH₂—, R₁ is substituted or unsubstituted 4-methoxyphenyl, 4-fluorophenyl, 2-fluorophenyl, 4-methylphenyl, 4-trifluoromethylphenyl, 4-pentafluoroethylphenyl, 4-methoxymethoxyphenyl, 4-hydroxyphenyl, 4-ethylphenyl, 1,3-benzodioxolyl, 1,4-benzodioxanyl or dihydrobenzofuranyl wherein the substituent is selected from alkoxy, alkoxyalkoxy and carboxyalkoxy, R₂ is 1,3-benzodioxolyl, 1,4-benzodioxanyl, dihydrobenzofuranyl, benzofuranyl, 4-methoxyphenyl, dimethoxyphenyl, fluorophenyl or difluorophenyl and R₃ is R₄—C(O)—R₅— wherein R₅ is alkylene and R₄ is (R₁₁)(R₁₂)N— wherein R₁₁ and R₁₂ are independently selected from loweralkyl.

Another most highly preferred embodiment of the invention is a compound of formula I or II wherein n is 0, R is —C(O)₂-G wherein G is hydrogen or a carboxy protecting group, Z is —CH₂—, R₁ is substituted or unsubstituted 4-methoxyphenyl, 4-fluorophenyl, 2-fluorophenyl, 4-methylphenyl, 4-trifluoromethylphenyl, 4-pentafluoroethylphenyl, 4-methoxymethoxyphenyl, 4-hydroxyphenyl, 4-ethylphenyl, 1,3-benzodioxolyl, 1,4-benzodioxanyl or dihydrobenzofuranyl wherein the substituent is selected from alkoxy, alkoxyalkoxy and carboxyalkoxy, R₂ is 1,3-benzodioxolyl, 1,4-benzodioxanyl, dihydrobenzofuranyl, benzofuranyl, 4-methoxyphenyl, dimethoxyphenyl, fluorophenyl or difluorophenyl and R₃ is R₄—C(O)—R₅— wherein R₅ is alkylene and R₄ is (R₁₁)(R₁₂)N— wherein R₁₁ is loweralkyl and R₁₂ is aryl.

Another most highly preferred embodiment of the invention is a compound of formula I or II wherein n is 0, R is —C(O)₂-G wherein G is hydrogen or a carboxy protecting group, Z is —CH₂—, R₁ is substituted or unsubstituted 4-methoxyphenyl, 3-fluoro-4-methoxyphenyl, 3-fluorophenyl, 2-fluorophenyl, 3-fluoro-4-ethoxyphenyl, 4-methoxymethoxyphenyl, 1,3-benzodioxolyl, 1,4-benzodioxanyl or dihydrobenzofuranyl wherein the substituent is selected from loweralkyl, haloalkyl, alkoxy, alkoxyalkoxy and carboxyalkoxy, R₂ is substituted or unsubstituted 1,3-benzodioxolyl, 7-methoxy-1,3-benzodioxolyl, 1,4-benzodioxanyl, 8-methoxy-1,4-benzodioxanyl, dihydrobenzofuranyl, 4-methoxyphenyl, dimethoxyphenyl, fluorophenyl or difluorophenyl wherein the substituent is selected from loweralkyl, alkoxy and halogen and R₃ is R₆—S(O)₂—N(R₂₁)—R₁₀— wherein R₁₀ is alkylene, R₆ is loweralkyl, haloalkyl, alkoxyalkyl or haloalkoxyalkyl and R₂₁ is loweralkyl, haloalkyl or alkoxyalkyl.

Another most highly preferred embodiment of the invention is a compound of formula I or II wherein n is 0, R is —C(O)₂-G wherein G is hydrogen or a carboxy protecting group, Z is —CH₂—, R₁ is substituted or unsubstituted 4-methoxyphenyl, 3-fluoro-4-methoxyphenyl, 3-fluorophenyl, 2-fluorophenyl, 3-fluoro-4-ethoxyphenyl, 4-methoxymethoxyphenyl, 1,3-benzodioxolyl, 1,4-benzodioxanyl or dihydrobenzofuranyl wherein the substituent is selected from loweralkyl, haloalkyl, alkoxy, alkoxyalkoxy and carboxyalkoxy, R₂ is substituted or unsubstituted 1,3-benzodioxolyl, 7-methoxy-1,3-benzodioxolyl, 1,4-benzodioxanyl, 8-methoxy-1,4-benzodioxanyl, dihydrobenzofuranyl, 4-methoxyphenyl, dimethoxyphenyl, fluorophenyl or difluorophenyl wherein the substituent is selected from loweralkyl, alkoxy and halogen and R₃ is R₄—C(O)—R₅— wherein R₅ is alkylene and R₄ is (R₁₁)(R₁₂)N— wherein R₁₁ is alkyl and R₁₂ is selected from aryl, aminoalkyl, trialkylaminoalkyl, and heterocyclic.

Another most highly preferred embodiment of the invention is a compound of formula I or II wherein n is 0, R is —C(O)₂-G wherein G is hydrogen or a carboxy protecting group, Z is —CH₂—, R₁ is loweralkyl,alkenyl, heterocyclic (alkyl), aryloxyalkyl, aryalkyl, aryl, (N-alkanoyl-N-alkyl)aminoalkyl, or alkylsulfonylamidoalkyl, and R₃ is R₄—C(O)—R₅— wherein R₅ is alkylene and R₄ is (R₁₁)(R₁₂)N— wherein R₁₁ and R₁₂ are independently selected from alkyl, aryl, hydroxyalkyl, alkoxy, aminoalkyl, trialkylaminoalkyl, and heterocyclic, with the proviso that one or R₁₁ and R₁₂ is alkyl.

Another most highly preferred embodiment of the invention is a compound of formula I or II wherein n is 0, Z is —CH₂—, and R₃ is R₄—C(O)—R₅— wherein R₄ is (R₁₁)(R₁₂)N— as defined therein and R₅ is alkylene.

Another most highly preferred embodiment of the invention is a compound of formula I or II wherein n is 0, Z is —CH₂—, R₁ is loweralkyl, and R₃ is R₄—C(O)—R₅— wherein R₄ is (R₁₁)(R₁₂)N— as defined therein and R₅ is alkylene.

Another most highly preferred embodiment of the invention is a compound of formula I or II wherein n is 0, Z is —CH₂—, R₁ is alkenyl, and R₃ is R₄—C(O)—R₅— wherein R₄ is (R₁₁)(R₁₂)N— as defined therein and R₅ is alkylene.

Another most highly preferred embodiment of the invention is a compound of formula I or II wherein n is 0, Z is —CH₂—, R₁ is heterocyclic (alkyl), and R₃ is R₄—C(O)—R₅— wherein R₄ is (R₁₁)(R₁₂)N— as defined therein and R₅ is alkylene.

Another most highly preferred embodiment of the invention is a compound of formula I or II wherein n is 0, Z is —CH₂—, R₁ is aryloxyalkyl, and R₃ is R₄—C(O)—R₅— wherein R₄ is (R₁₁)(R₁₂)N— as defined therein and R₅ is alkylene.

Another most highly preferred embodiment of the invention is a compound of formula I or II wherein n is 0, Z is —CH₂—, R₁ is arylalkyl, and R₃ is R₄—C(O)—R₅— wherein R₄ is (R₁₁)(R₁₂)N— as defined therein and R₅ is alkylene.

Another most highly preferred embodiment of the invention is a compound of formula I or II wherein n is 0, Z is —CH₂—, R₁ is aryl, and R₃ is R₄—C(O)—R₅— wherein R₄ is (R₁₁)(R₁₂)N— as defined therein and R₅ is alkylene.

Another most highly preferred embodiment of the invention is a compound of formula I or II wherein n is 0, Z is —CH₂—, R₁ is (N-alkanoyl-N-alkyl)aminoalkyl, and R₃ is R₄—C(O)—R₅— wherein R₄ is (R₁₁)(R₁₂)N— as defined therein and R₅ is alkylene.

Another most highly preferred embodiment of the invention is a compound of formula I or II wherein n is 0, Z is —CH₂—, R₁ is alkylsulfonylamidoalkyl, and R₃ is R₄—C(O)—R₅— wherein R₄ is (R₁₁)(R₁₂)N— as defined therein and R₅ is alkylene.

A particularly preferred compound of formula I is a compound of formula III, also known as ABT-627:

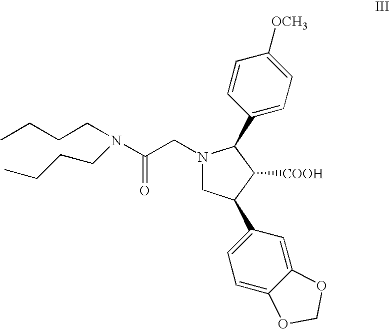

Compounds of formula I, II, and III may be synthesized by methods provided in commonly owned U.S. patent application Ser. No. 09/048,955, filed Mar. 27, 1998, U.S. patent application Ser. No. 08/794,506, filed Feb. 4, 1997, U.S. patent application Ser. No. 08/600,625, filed Feb. 13, 1996, U.S. patent application Ser. No. 08/497,998, filed Aug. 2, 1995, U.S. patent application Ser. No. 08/442,575, filed May 30, 1995, U.S. patent application Ser. No. 08/334,717, filed Nov. 4, 1994, and U.S. patent application Ser. No. 08/293,349, filed Aug. 19, 1994, the disclosures of which are herein incorporated by reference.

Other suitable endothelin ET-A receptor antagonist may be used, such as those disclosed in U.S. Pat. Nos. 6,048,893, 6,017,951, and 5,998,468.

The term “inhibit” is defined to include its generally accepted meaning which includes preventing, prohibiting, restraining, and slowing, stopping or reversing progression, or severity, and holding in check and/or treating existing characteristics. The present method includes both medical therapeutic and/or prophylactic treatment, as appropriate.

The methods of the present invention are useful in men as well as in women. Preferably, however, the methods of the present invention are useful in men, more preferably men with prostate cancer.

The ability of the compounds of formula I, II, and III to treat cancers can be demonstrated according to the method described in J. Clin. Invest. 87 1867 (1991). Types of cancer includes primary cancer such as breast, prostate, lung, kidney, thyroid, myeloma, lymphoma, sarcoma, osteosarcoma, and ovarian.

The ability of the compounds of the invention to treat nociception can be demonstrated according to the method described in J. Pharmacol. Exp. Therap. 271 156 (1994).

The compounds of the present invention can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, p-toluenesulfonate and undecanoate. Also, the basic nitrogen-containing groups can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid.

Basic addition salts can be prepared in situ during the final isolation and purification of the compounds of formula I, or separately by reacting the carboxylic acid function with a suitable base such as the hydroxide, carbonate or bicarbonate of a pharmaceutically acceptable metal cation or with ammonia, or an organic primary, secondary or tertiary amine. Such pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

The compounds of formulas I, II and III are useful for antagonizing endothelin in humans or other mammals.

Total daily dose administered to a host in single or divided doses may be in amounts, for example, from 0.001 to 1000 mg/kg body weight daily and more usually 0.1 to 100 mg/kg for oral administeration or 0.01 to 10 mg/kg for parenteral administeration. Dosage unit compositions may contain such amounts of submultiples thereof to make up the daily dose.

Pharmaceutical formulations may be prepared by procedures known in the art. The amount of active ingredient that may be combined with the carrier materials to produce a single dosage form will vary depending upon the host treated and the particular mode of administeration.

It will be understood, however, that the specific dose level for any particular patient will depend upon a variety of factors including the activity of the specific compound employed, the age, body weight, general health, sex, diet, time of administeration, route of administeration, rate of excretion, drug combination, and the severity of the particular disease undergoing therapy.

The compounds of the present invention may be administered orally, buccally, parenterally, sublingually, by inhalation spray, rectally, or topically in dosage unit formulations containing conventional nontoxic pharmaceutically acceptable carriers, adjuvants, and vehicles as desired. Topical administeration may also involve the use of transdermal administeration such as transdermal patches or iontophoresis devices. The term parenteral as used herein includes subcutaneous injections, intravenous, intramuscular, intrasternal injection, transcutaneous, intradermal, or infusion techniques.

Injectable preparations, for example, sterile injectable aqueous or oleagenous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administeration of the drug can be prepared by mixing the drug with a suitable nonirritating excipient such as cocoa butter and polyethylene glycols which are solid at ordinary temperatures but liquid at the rectal temperature and will therefore melt in the rectum and release the drug.

Solid dosage forms for oral administeration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administeration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic.

Methods to form liposomes are known in the art. See, for example, Prescott, Ed., Methods in Cell Biology, Volume XIV, Academic Press, New York, N.Y. (1976), p. 33 et seq.

A representative solid dosage form, for example, a tablet or a capsule, comprises:

| | |
|---|---|
| Compound of the invention: | 35% w/w |
| Starch, Pregelatinized, NF | 50% w/w |
| Microcrystalline Cellulose, NF | 10% w/w |
| Talc, Powder, USP | 5% w/w |

While the compounds of the invention can be administered as the sole active therapeutic agent, they can also be used in combination with one or more co-therapeutic agents, such as anticancer drugs or methods including, but not limited to, hormonal agents, such as leuprolide (Lupron®); gonadorelin antagonists, such as goserelin (Zoladex®) and abarelix; bicalutamide; nilutamide; flutamide; vitamin D; vitamin D analogues; estrogen and estrogen analogues, such as diethylstibestrol; prednisone; hydrocortisone; ketoconazole; cyproterone acetate; progesterone; 5-alpha reductase inhibitors, such as finasteride; bone-seeking radionuclides, such as samarium (Quadramet®), strontium (Metastron®), and ¹⁸⁶rhenium; external beam radiation, including three dimensional conformal radiation; brachytherapy, which is the implantation of radioactive seeds directly into the prostate; monoclonal antibodies such as trastuzumab (Herceptin®); anti-angiogenic agents such as thrombospondin peptide or kringle 5; matrix metalloproteinase inhibitors; farnesyl transferase inhibitors; lycopenes; urokinase; plasminogen activator inhibitors; plasminogen activator receptor blockers; apoptosis inducers; selective and non-selective alpha blockers; platinum agents, such as cis-platinum and carbo-platinum; taxane class agents, such as docitaxil and paclitaxil; estramustine; gemcytabine; adriamycin; doxorubicin; daunorubicin; mitoxantrone; vinblastine; vincristine; capecitabine; irinotecan; topotecan; 5-fluorouracil; interferons; cytoxan; methotrexate; cytokines, such as IL-2; PPAR agonists, such as thiazolidine diones; retinoid-type agents, 5-lipooxygenase inhibitors, such as zyfo (Zilueton®), COX-2 inhibitors; gene-therapy based therapeutics, including sense and anti-sense genes; cholesterol lowering drugs, such as lovastatin, pravastatin, and simvistatin; bisphosphonates; osteoprotegrin; and antibodies, both monoclonal and polyclonal; antibody-coupled radionucleotides; antibody-coupled cytotoxic agents; antibody-coupled radionucleotides; viral-vector delivered agents; vaccines directed at protein, carbohydrate, or nucleic acid targets; aminoglutethimide; and suramin.

These combinations can be administered as separate compositions or as a single dosage form containing both or all agents. When administered as a combination, the therapeutic agents can be formulated as separate compositions, which are given at the same time or different times, or the therapeutic agents can be given as a single composition.

In addition, the compounds invention can be used in combination with one or more co-therapeutic agents which impede net bone loss, such as estrogens, bisphosphonates, and estrogen receptor modulators, such as raloxifene, and calcitonin.

The compounds of the invention can additionally be administered in combination with surgery, such as radical prostatectomy, cryotherapy, transurethral resection of the prostate as an adjuvant, and the like, or prior to surgery as a neoadjuvant agent.

The current major diseases or conditions of bone which are of public concern include, but are not limited to, post-menopausal osteoporosis, ovariectomy patients, senile osteoporosis, patients undergoing long-term treatment of corticosteroids, side effects from glucocorticoid or steroid treatment, patients suffering from Cushings's syndrome, gonadal dysgenesis, periarticular erosions in rheumatoid arthritis, osteoarthritis, Paget's disease, osteohalisteresis, osteomalacia, hypercalcemia of malignancy, osteopenia due to bone metastases, periodontal disease, hyperparathyroidism, osteroperosis from Lupron therapy, and starvation. All of these conditions are characterized by bone loss, resulting from an imbalance between the degradation of bone (bone resorption) and the formation of new healthy bone. This turnover of bone continues normally throughout life and is the mechanism by which bone regenerates. However, the conditions stated above will tip the balance towards bone loss such that the amount of bone resorbed is inadequately replaced with new bone, resulting in net bone loss.

### EXAMPLES

Studies were performed on male subjects with asymptomatic hormone refractory prostate cancer with rising PSA levels and on male subjects with symptomatic hormone refractory prostate cancer with rising PSA levels and pain. Subjects in the phase II studies had castrate levels of testosterone, either due to pharmacologic intervention, via leuprolide (Lupron®) or goserelin (Zoladex®), or via surgical castration. Subjects received ABT-627 or placebo. The following tests were conducted:

ABT-627 was formulated in 2.5 and 10 mg doses. An oral liquid formulation of ABT-627 was also prepared as follows: 1 mg/ml ABT-627, 50% glycerin, 14% alcohol, and water. Matching placebos were also provided.

A number of recognized or putative biochemical markers of disease progression have been used to monitor treatment of individuals with prostate cancer. Among these markers are serum Prostate Specific Antigen (PSA), serum acid Phosphatase, Interleukin-6, and Chromagranin-A. As currently accepted, favorable treatment is marked by a decrease or slower rate of increase for PSA, acid phosphatase, and Interleukin-6, while a favorable response is marked by an increase in Chromagranin-A.

Serum samples were obtained from subjects during treatment with the ET antagonist ABT-627 in order to determine PSA, acid phosphatase, IL-6, and Chromagranin-A values.

### Prostate Specific Antigen Level Assay

The effect of ABT-627 administeration on prostate specific antigen (PSA) levels in human subject serum samples was determined using the procedure described in the Chiron Diagnostics ACS: Centaur PSA2 Assay. This assay is a two-site sandwich immunoassay which uses direct chemiluminescense and constant amounts of two antibodies. The first antibody, the Lite Reagent, is an affinity purified polyclonal sheep anti-PSA antibody labeled with acridinium ester. The Lite Reagent is purchased as a 5.0 mL reagent pack comprising the polyclonal sheep anti-PSA antibody (3.1 μg) in buffered saline with sodium azide (0.1%). The second antibody, the Solid Phase, is a monoclonal mouse anti-PSA antibody covalently coupled to paramagnetic particles. The Solid Phase is purchased as a 25.0 mL reagent pack comprising the covalently coupled monoclonal mouse anti-PSA antibody (316 μg) in buffered saline with sodium azide (0.1%). The assay was performed at Quintiles Laboratories (Smyrna, Ga.) using Chiron Diagnostics ACS: Centaur® Automated Chemiluminescence Systems.

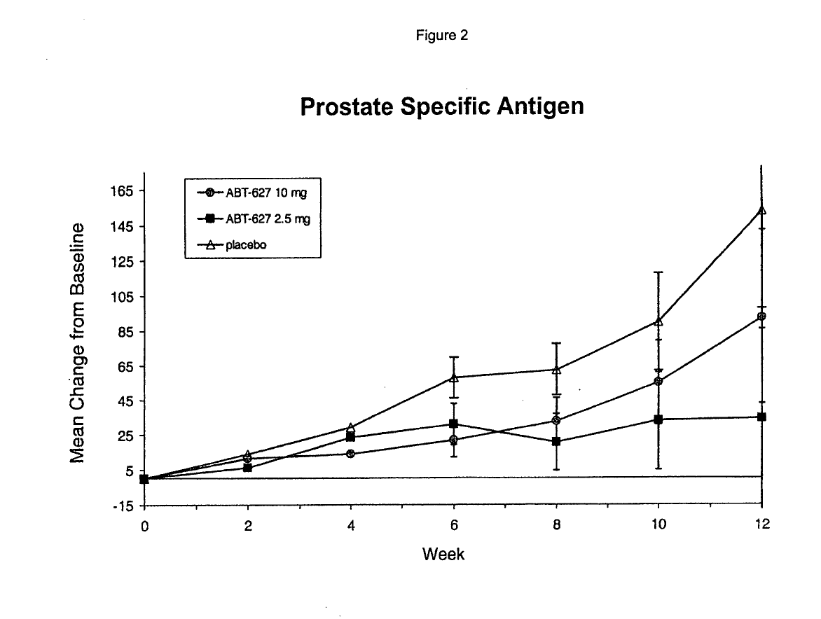

Briefly, a subject population was treated with a placebo or 2.5 mg or 10 mg of ABT-627. Blood samples were collected, allowed to adequately clot, centrifuged at 1000×g for 15-20 minutes, and stored at −20° C. if not assayed within 48 hours. A cuvette was charged sequentially with serum, Lite Reagent (50 μL), and Solid Phase (250 μL). The resulting mixture was incubated for 7.5 minutes at 37° C., separated, and treated with the solution of Acid Reagent and Base Reagent to initiate the chemiluminescent reaction. A direct relationship exists between the amount of PSA present in the patient sample and the RLU's (relative light units) detected. As shown by the area under the curve (AUC) in FIG. 2, the rate of increase of PSA in the serum samples decreases after the adminsteration of ABT-627, demonstrating the effectivness of ABT-627 as an agent for treating prostate cancer.

### Acid Phosphatase Levels

The effect of ABT-627 administeration on Acid Phosphatase levels in human subject serum samples was determined at Quintiles Laboratories using the chemical test described in Sigma Diagnostics Acid Phosphatase (ACP) Procedure No. 435. The enzyme Acid Phosphatase (ACP) catalyzes the hydrolysis of alpha-naphthyl phosphate to alpha-naphthol and inorganic phosphate. The alpha-naphthol immediately reacts with fast red TR salt to produce a yellow chromophore with an absorbance maximum at 405 nm. The rate of increase in absorbance at 405 nm is directly proportional to ACP activity in the sample. ACP activity was determined in the presence and absence of L-tartrate, the difference being attributed to prostatic acid phosphatase activity.

Briefly, a subject population was treated with a placebo or 2.5 mg or 10 mg of ABT-627. Blood samples were collected, allowed to adequately clot, centrifuged at 1000×g for 15-20 minutes, and stored at −20° C. if not assayed within 48 hours. Assays were performed on a Hitachi Spectrophotomer. A cuvette was charged sequentially with ACP reagent (1 mL), prepared as described in the assay protocol, and serum (0.1 mL). The mixture was agitated and incubated for 5 minutes, and an absorbance (A) at 405 nm (versus water as a reference) was read to provide an initial absorbance. The mixture was incubated for another 5 minutes, and a second absorbance was read to provide a final absorbance. A change A/5 minute value was obtained by subtracting the initial absorbance from the final absorbance and was used to calculate total ACP activity.

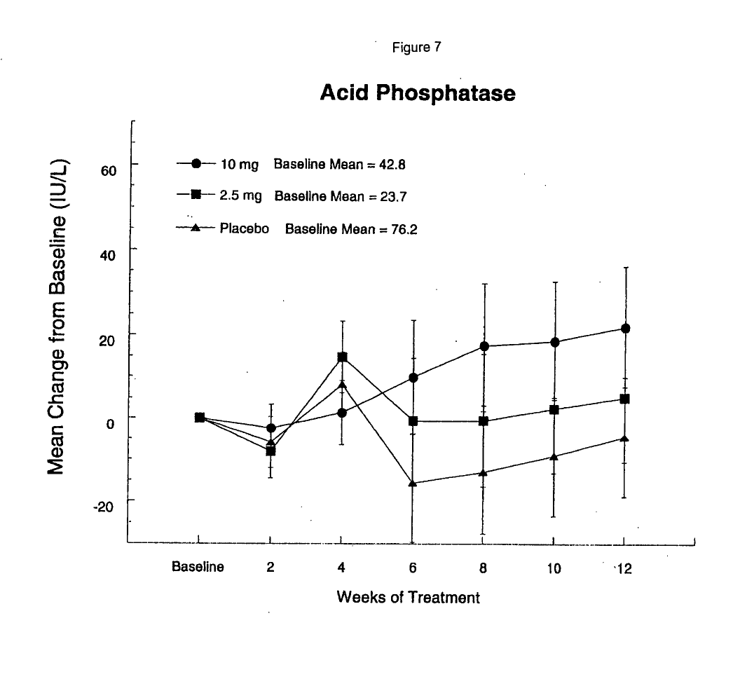

To provide the tartrate-resistant acid phosphatase activity, the above procedure was repeated with the addition of ACP tartrate reagent (0.01 mL) to the cuvette containing the ACP reagent and mixing before adding the serum. Prostatic acid phosphatase activity was calculated by subtracting the the tartrate-resistant acid phosphatase activity from the ACP activity. As shown shown by the (AUC) in FIG. 7, the rate of increase and the average change from baseline for acid phosphatase was decreased in those subjects treated with ABT-627, again demonstrating the effectivness of ABT-627 as an agent for treating prostate cancer.

### Chromagranin-A Levels

The effect of ABT-627 adminstration on Chromagranin-A levels in human serum samples was determined by proprietary assay conducted at the Nichols Institute. The procedure is a two site chemiluminescence assay (ICMA) using one monoclonal antibody conjugated with biotin, another monoclonal antibody labeled with an acridinium ester, and an avidin-coated solid phase. The antibody/Chromagranin-A/antibody complex is bound to the solid phase by the avidin-biotin interaction and unbound materials are removed by washing. The bound, acridinium-labeled material produces light that is detected in a luminometer after addition of triggering agents. The Limit of Detection (LOD) for the assay was 0.07 ng/mL. As shown by the AUC in FIG. 8, the average change from baseline for Chromagranin-A was higher for subjects treated with 2.5 mg/day of ABT-627, again demonstrating the effectivness of ABT-627 as an agent for treating prostate cancer.

### Interleukin-6 Levels

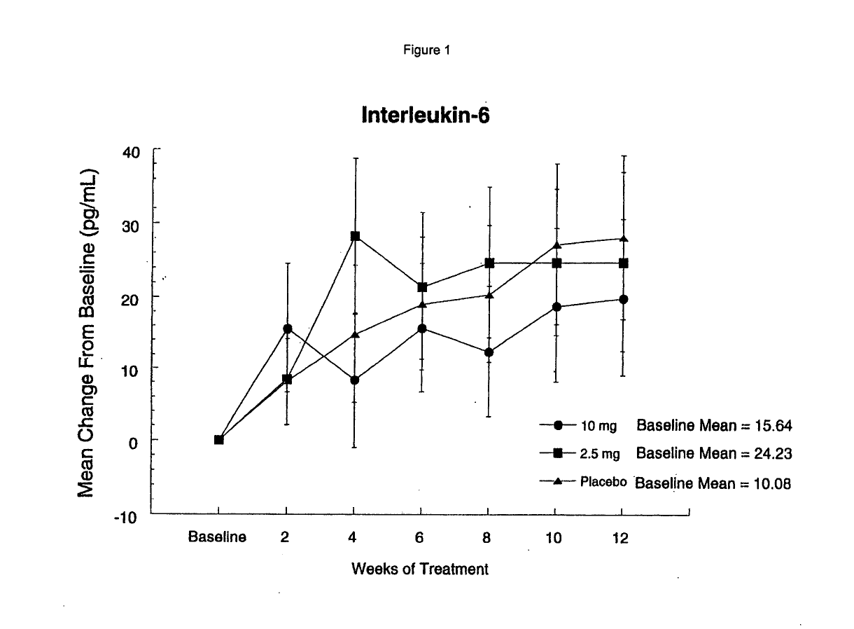

The effect of ABT-627 adminstration on Interleukin-6 levels in human serum samples was determined at Quintiles Laboratories using a sandwich immunoassay. Human serum samples and standards were incubated in microtiter plate wells coated with a monoclonal anti-IL-6 antibody, in the presence of a second monoclonal anti IL-6 antibody, linked to acetylcholinesterase. After incubation, the wells were washed, and the bound enzymatic activity was measured using a chromogenic substrate. The intensity of the color was proportional to the concentration of IL-6 in the sample or standard. As shown by the AUC FIG. 1, the average change in baseline for Interleukin-6 was lower in those subjects treated with ABT-627, demonstrating the effectivness of ABT-627 as an agent for reducing inflammation and ameliorating pain.

### Bone Scan Methodology

Bone scans were performed with an NDA approved, Tc-99m phosphonate type radiopharmaceutical. This technique uses whole body format (skull to feet) so that anterior and posterior images are presented when using a 510 K-approved gamma camera. Alternatively, spot views covering both anterior and posterior projections of the total body can be obtained. Interpretation was performed according to standard nuclear medicine criteria, on a bone by bone basis, by recording the number of lesions at each site. Each site was evaluated against a confidence score of 1 to 5, where 1 is negative, 2 is probably negative, 3 is equivocal, 4 is probably positive, and 5 is definitely positive. The MSKCC (Clin. Can. Res. 1998; 4:1765-1772) was used to record these findings. For the purposes of scoring the extent of disease or the response to treatment, lesions with a confidence score of 4 and 5 were considered positive, and all other lesions were considered negative. In addition, in a blinded study, a reference nuclear medicine physician interpreted the bone scans quantitatively as follows: the percent of involved bone was estimated for each individual bone, and the individual bone involvement was summed to calculate a global percent bone scan index (BSI). More specifically, the bone scan was separated into three indices. The first was the appindicular scan which involved arms and legs (i.e. the humorous and all bones distal to the humerous and the femur and everything distal to the femur). The second was the axial (everything but the arms and the legs). The results of these scans were combined to provide the total BSI.

Bone scans were conducted on each subject on day one of the study, and on the final day of the study, and the changes from baseline in bone scan index scores were analysed by mean change and mean percent change, adjusting for baseline characteristics as co-variates using SAS version XXX software.

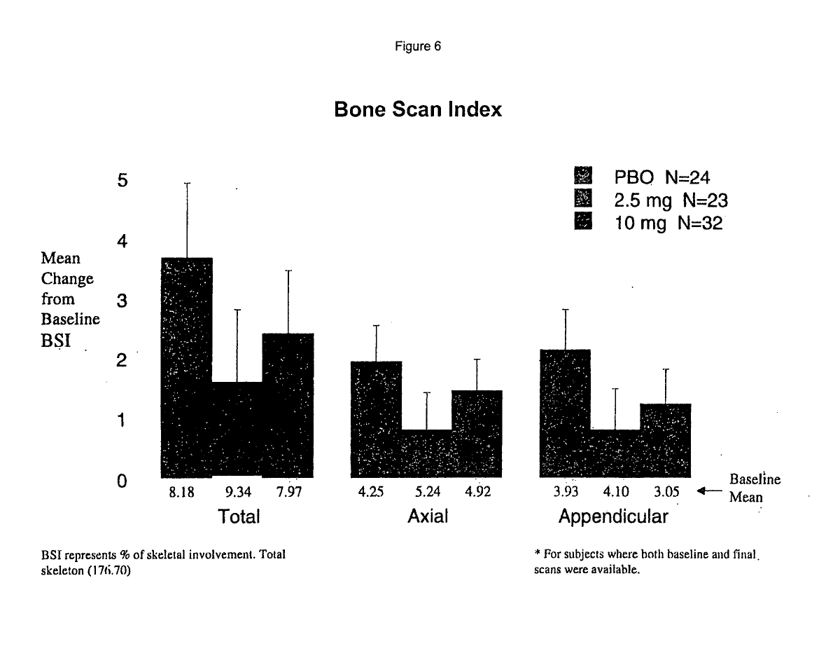

As shown in FIG. 6, bone scans indicated a decrease in the proportion of total skeketal involvement in those subjects receiving ABT-627 versus placebo, demonstrating the effectivness of ABT-627 as an agent for reducing the fraction of total skeletal involvement by tumor.

### VAS Methodology/Administeration/Analysis

The Visual Analog Scale (VAS) is a common instrument of pain assessment performed by having a subject draw a vertical line on a 10 cm scale at the point that best describes his or her pain on average in the last 24 hours. A diagram of the scale is shown below:

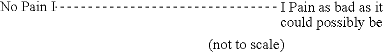

During the course of the study, pain asessments were done daily, at bedtime, by the subject. If the subject was unable to maintain the log, a caregiver could complete the log on his or her behalf. The log also contained a table on which was recorded all daily pain medication consumed by the patient. The logs of daily VAS scores and analgesic consumption were collected at biweekly visits of the subject to the clinic when a new log was distributed. Clinical personnel who received the logs measured the score by measuring the distance (in mm) from the “no pain” end mark to the point where the subject's line crossed the VAS line. The number was written into the case report form next to the date the subject completed that page of the logbook.

Subjects with pain were initially stabilized in their pain so that their pain was treated to a tolerable and constant level. For this study, “tolerable and constant” refers to a pain score less than or equal to 5 cm on the VAS for an average of seven successive days while using four or less rescue doses of pain medication per day. A rescue medication dose refers to a dose equal to one single dose a patient used for common timed pain relief.

The weekly VAS scores were calculated excluding the lowest and highest score for each week and averaging the remaining five scores. If there were two days with the same VAS score, the day with the highest analgesic use was discarded.

The weekly mean VAS score was used to define subjects as responders or non-responders. A subject was considered a responder based on the reduction in the pain intensity: a weekly VAS score reduction of greater than or equal to 25% during at least two consecutive weeks without an increase of analgesic use during the same period (compared to baseline). Alternatively, a subject was considered a responder if his pain analgesic consumption was reduced by at least 25% during at least two consecutive weeks without a concomitant increase in VAS score.

The percentage of responders in each treatment group was compared to evaluate drug efficacy. The comparison was subjected to an adjustment for baseline characteristics and prognostic factors as co-variates, and the analysis was performed using the Cochran-Mantel-Haenszel test or a generalized linear model.

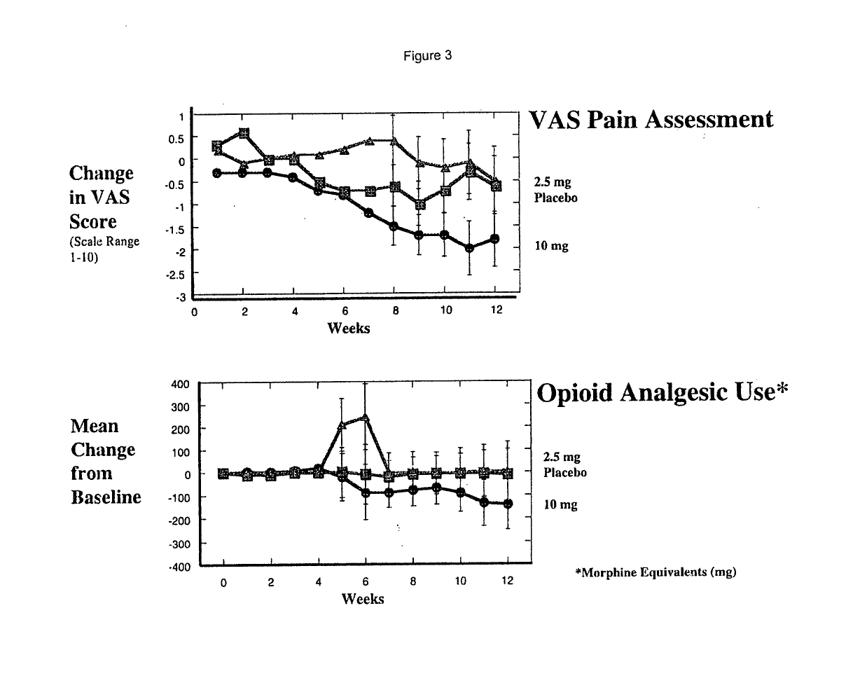

Weekly VAS scores are examined using a longitudinal analysis method to explore trends over time. The duration of the response, defined as the time from baseline to the last weekly assessment for which the responder definition was satisfied, was analyzed using the Kaplan-Meier methodology and logrank test. Cox proportional hazard models were used as needed (see U.S. Department of Health and Human Services. Management of Cancer Pain Clinical Practice Guidelines. AHCPR Publication #94-0592, Rockville, Md. (1994). As shown by the AUC in FIG. 3, VAS scores showed a decrease in pain, independent of the effects of morphine, after treatment with with ABT-627, demonstrating the effectivness of ABT-627 as an agent for ameliorating pain.

### Osteoblastic Activity and Bone Markers

Markers of osteoblastic activity were assessed using urine samples. Bone markers include bone alkaline phosphatase (BAP), deoxypridinoline, and N-telopeptide of Type I collagen. Blood samples were collected prior to dosing on Day 1, Day 42, Day 84, Day 168, and every 28 days after Day 168, with a final collection on the last day of the study.

### Bone Alkaline Phosphatase

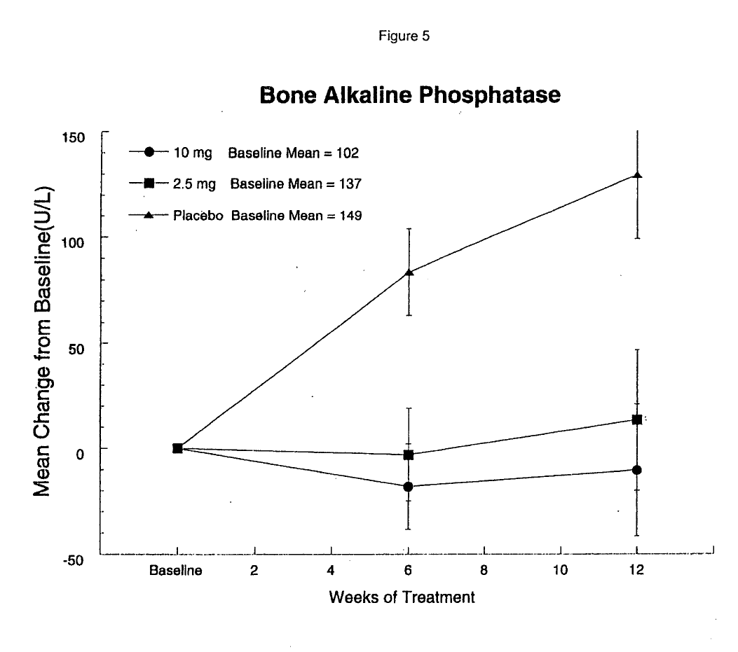

Bone Alkaline Phosphatase levels were determined using the bone-specific Alkphase-B® assay published by Metra Biosystems (Mountain View, Calif.). As shown by the AUC in FIG. 5, BAP levels decreased in subjects treated with ABT-627, demonstrating the effectivness of ABT-627 as an agent for inhibiting abnormal bone remodeling.

### Crosslinked N-Telopeptide Levels

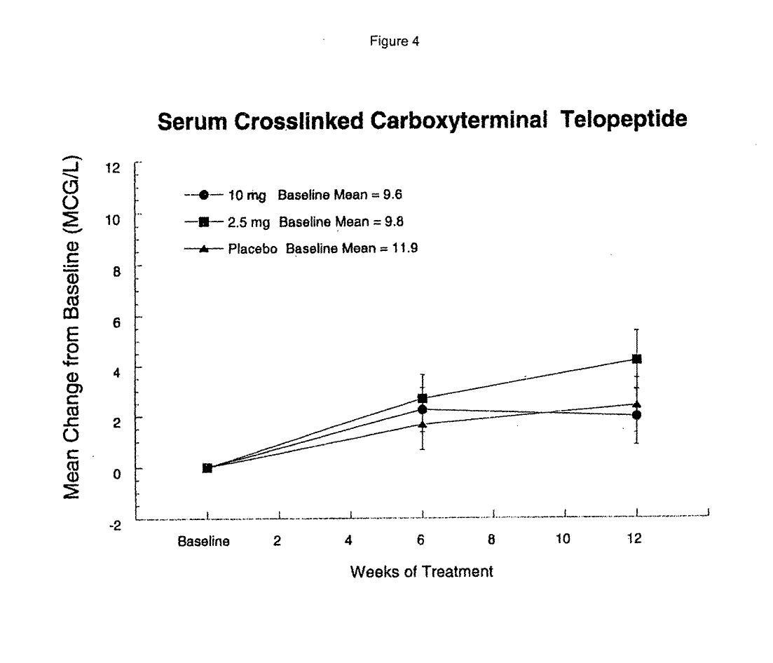

Cross-linked N-telopeptide levels were determined using the DiaSorin (Stillwater, Minn.) assay for the quantitative determination of carboxyterminal cross-linked telopeptide of type I collagen (ICTP) in human serum by equilibrium radioimmunoassay (RIA). Briefly, samples were incubated with the ¹²⁵I ICTP tracer and ICTP primary antibody for 2 hours at 37° C. Following the 2 hour incubation, a pre-precipitated second antibody complex was added to separate the bound from free tracer. The assay was then centrifuged and decanted after a 30 minute incubation at room temperature. The bound tracer in the pellet was counted with a gamma counter. Counts were inversely proportional to the amount of ICTP present in each sample. As shown by the AUC in FIG. 4, Crosslinked N-telopeptide levels decreased in subjects treated with ABT-627, demonstrating the effectivness of ABT-627 as an agent for inhibiting the bone remodeling associated with bone diseases.

1-109. (canceled)

110. A method for inhibiting bone metastases from prostate cancer in a human comprising administering thereto a therapeutically effective amount of the compound of formula III 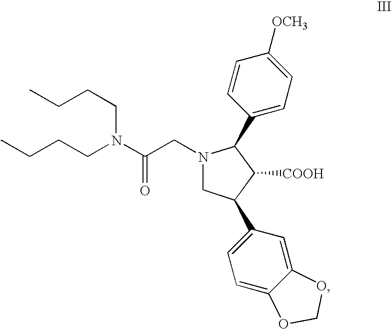
or a pharmaceutically acceptable salt thereof.

111. The method of claim 110 wherein the bone metastases are osteoblastic.

112. The method of claim 110 which additionally comprises administering an anticancer drug.

113. The method of claim 112 wherein the anticancer drug is selected from the group consisting of leuprolide, goserelin, bicalutamide, nilutamide, flutamide, vitamin D, vitamin D analogues, estrogen, estrogen analogues, prednisone, hydrocortisone, ketoconazole, cyproterone acetate, and progesterone.

114. The method of claim 10 which additionally comprises administering radiation.

115. The method of claim 110 which additionally comprises administering at least one therapeutic agent which impedes net bone loss.

116. The method of claim 115 wherein the therapeutic agent is a bisphosphonate.

117. A method for inhibiting metastatic growth from prostate cancer in a human comprising administering thereto a therapeutically effective amount of the compound of formula III 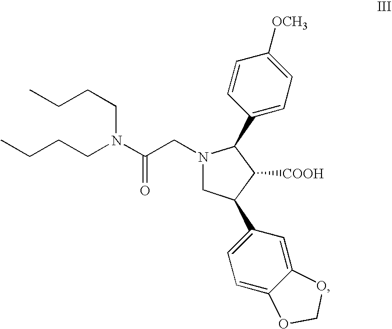
or a pharmaceutically acceptable salt thereof.

118. The method of claim 117 wherein the metastatic growth is osteoblastic metastatic growth.

119. The method of claim 117 which additionally comprises administering an anticancer drug.

120. The method of claim 119 wherein the anticancer drug is selected from the group consisting of leuprolide, goserelin, bicalutamide, nilutamide, flutamide, vitamin D, vitamin D analogues, estrogen, estrogen analogues, prednisone, hydrocortisone, ketoconazole, cyproterone acetate, and progesterone.

121. The method of claim 117 which additionally comprises administering radiation.

122. The method of claim 117 which additionally comprises administering at least one therapeutic agent which impedes net bone loss.

123. The method of claim 122 wherein the therapeutic agent is a bisphosphonate.

124. A method for inhibiting osteoblastic bone metastases from prostate cancer in a human comprising administering thereto a therapeutically effective amount of the compound of formula III 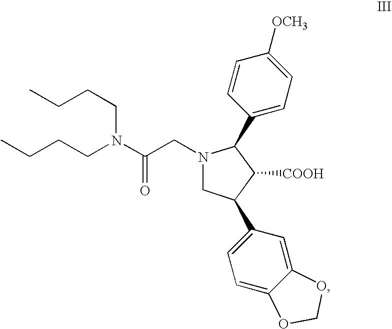
or a pharmaceutically acceptable salt thereof.

125. The method of claim 124 which additionally comprises administering an anticancer drug.

126. The method of claim 125 wherein the anticancer drug is selected from the group consisting of leuprolide, goserelin, bicalutamide, nilutamide, flutamide, vitamin D, vitamin D analogues, estrogen, estrogen analogues, prednisone, hydrocortisone, ketoconazole, cyproterone acetate, and progesterone.

127. The method of claim 124 which additionally comprises administering radiation.

128. The method of claim 124 which additionally comprises administering at least one therapeutic agent which impedes net bone loss.

129. The method of claim 128 wherein the therapeutic agent is a bisphosphonate.

130. A method for inhibiting osteoblastic metastatic growth from prostate cancer in a human comprising administering thereto a therapeutically effective amount of the compound of formula III 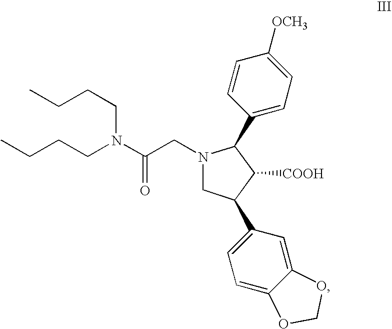
or a pharmaceutically acceptable salt thereof.

131. The method of claim 130 which additionally comprises administering an anticancer drug.

132. The method of claim 131 wherein the anticancer drug is selected from the group consisting of leuprolide, goserelin, bicalutamide, nilutamide, flutamide, vitamin D, vitamin D analogues, estrogen, estrogen analogues, prednisone, hydrocortisone, ketoconazole, cyproterone acetate, and progesterone.

133. The method of claim 130 which additionally comprises administering radiation.

134. The method of claim 130 which additionally comprises administering at least one therapeutic agent which impedes net bone loss.

135. The method of claim 134 wherein the therapeutic agent is a bisphosphonate.

136. A method for inhibiting bone metastases from prostate cancer in a human comprising administering thereto a therapeutically effective amount of the hydrochloride salt of the compound of formula III

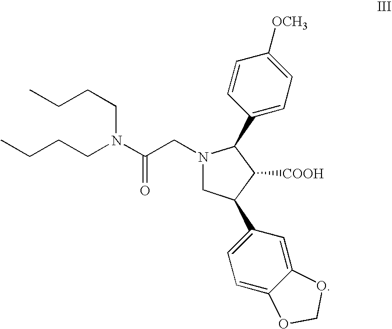

137. The method of claim 136 wherein the bone metastases are osteoblastic.

138. The method of claim 136 which additionally comprises administering an anticancer drug.

139. The method of claim 136 wherein the anticancer drug is selected from the group consisting of leuprolide, goserelin, bicalutamide, nilutamide, flutamide, vitamin D, vitamin D analogues, estrogen, estrogen analogues, prednisone, hydrocortisone, ketoconazole, cyproterone acetate, and progesterone.

140. The method of claim 136 which additionally comprises administering radiation.

141. The method of claim 136 which additionally comprises administering at least one therapeutic agent which impedes net bone loss.

142. The method of claim 141 wherein the therapeutic agent is a bisphosphonate.

143. A method for inhibiting metastatic growth from prostate cancer in a human comprising administering thereto a therapeutically effective amount of the hydrochloride salt of the compound of formula III

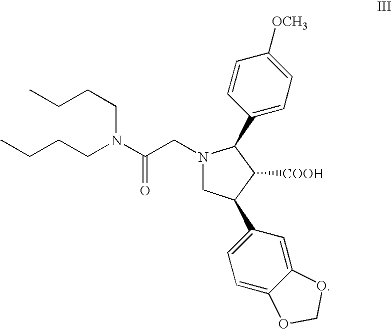

144. The method of claim 143 wherein the metastatic growth is osteoblastic metastatic growth.

145. The method of claim 143 which additionally comprises administering an anticancer drug.

146. The method of claim 145 wherein the anticancer drug is selected from the group consisting of leuprolide, goserelin, bicalutamide, nilutamide, flutamide, vitamin D, vitamin D analogues, estrogen, estrogen analogues, prednisone, hydrocortisone, ketoconazole, cyproterone acetate, and progesterone.

147. The method of claim 143 which additionally comprises administering radiation.

148. The method of claim 143 which additionally comprises administering at least one therapeutic agent which impedes net bone loss.

149. The method of claim 148 wherein the therapeutic agent is a bisphosphonate.

150. A method for inhibiting osteoblastic bone metastases from prostate cancer in a human comprising administering thereto a therapeutically effective amount of the hydrochloride salt of the compound of formula III

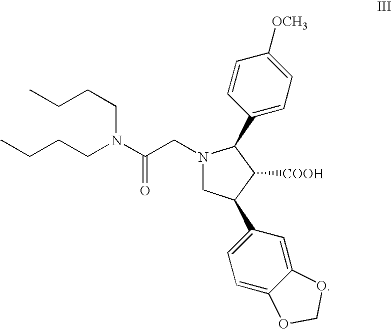

151. The method of claim 150 which additionally comprises administering an anticancer drug.

152. The method of claim 151 wherein the anticancer drug is selected from the group consisting of leuprolide, goserelin, bicalutamide, nilutamide, flutamide, vitamin D, vitamin D analogues, estrogen, estrogen analogues, prednisone, hydrocortisone, ketoconazole, cyproterone acetate, and progesterone.

153. The method of claim 150 which additionally comprises administering radiation.

154. The method of claim 150 which additionally comprises administering at least one therapeutic agent which impedes net bone loss.

155. The method of claim 154 wherein the therapeutic agent is a bisphosphonate.

156. A method for inhibiting osteoblastic metastatic growth from prostate cancer in a human comprising administering thereto a therapeutically effective amount of the hydrochloride salt of the compound of formula III

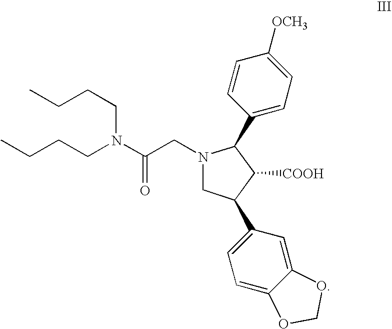

157. The method of claim 156 which additionally comprises administering an anticancer drug.

158. The method of claim 157 wherein the anticancer drug is selected from the group consisting of leuprolide, goserelin, bicalutamide, nilutamide, flutamide, vitamin D, vitamin D analogues, estrogen, estrogen analogues, prednisone, hydrocortisone, ketoconazole, cyproterone acetate, and progesterone.

159. The method of claim 156 which additionally comprises administering radiation.

160. The method of claim 156 which additionally comprises administering at least one therapeutic agent which impedes net bone loss.

161. The method of claim 160 wherein the therapeutic agent is a bisphosphonate.
